(19) United States
(12) Patent Application Publication
(10) Pub. No.: US 2025/0288706 A1
(43) Pub. Date: Sep. 18, 2025

(54) Automatic Tray Table Disinfection for Tray Tables in an Aircraft

(71) Applicant: The Boeing Company, Arlington, VA (US)

(72) Inventors: Nathan D. Hiller, Irvine, CA (US); Claudia E. Fritz-Thompson, Bothell, WA (US)

(21) Appl. No.: 19/058,819

(22) Filed: Feb. 20, 2025

Related U.S. Application Data
(60) Provisional application No. 63/564,303, filed on Mar. 12, 2024.

(51) Int. Cl. A61L 2/10; A61L 2/08; B64D 11/00; B64D 11/06

(52) U.S. Cl. CPC A61L 2/10; A61L 2/085; B64D 11/0638; A61L 2202/11; A61L 2202/20; B64D 2011/0053

(57) ABSTRACT

Automatic tray table disinfection system comprises a light system configured to emit a light that illuminates a surface of a tray table in a stored position. The light has a set of characteristics that disinfects the surface of the tray table.

### CROSS-REFERENCE TO RELATED APPLICATION

This application claims the benefit of U.S. Provisional Patent Application Ser. No. 63/564,303, filed Mar. 12, 2024, and entitled “Automatic Tray Table Sterilization for Tray Tables in an Aircraft,” which is incorporated herein by reference in its entirety.

### BACKGROUND INFORMATION

### 1. Field

The present disclosure relates generally to aircraft and in particular, to disinfecting tray table surfaces in the passenger cabin of an aircraft.

### 2. Background

A tray table in a passenger cabin of an aircraft likely has more germs than a toilet seat or a mobile phone. For example, tray tables can have more than 2,000 colony-forming units of bacteria per square inch. In contrast, studies have shown that mobile phones can have 27 to 30 colony-forming units per square inch.

Pre or post travel disinfection of the tray tables can be performed by cleaning crews using hand wipes or spraying disinfectant. These actions help reduce bacteria colony counts and other microbes. However, this type of disinfection process is time consuming and labor intensive.

### SUMMARY

An embodiment of the present disclosure provides an automatic tray table disinfection system comprising a light system configured to emit a light that illuminates a surface of a tray table in a stored position. The light has a set of characteristics that disinfects the surface of the tray table.

Another embodiment of the present disclosure provides a method for disinfecting a tray table. The tray table is detected in a stored position. A light is emitted to illuminate a surface of the tray table in the stored position. The light has a set of characteristics that disinfects the surface of the tray table.

Yet another embodiment of the present disclosure provides an automatic tray table heating system comprising a light system configured to emit a light that illuminates a surface of a tray table in a stored position, wherein the light has a set of characteristics that heats the surface of the tray table.

Still another embodiment of the present disclosure provides a method for heating a surface of a tray table. The tray table is detected in a stored position. A light is emitted to illuminate the surface of the tray table in the stored position, wherein the light has a set of characteristics that heats the surface of the tray table.

Another embodiment of the present disclosure provides an automatic tray table lighting system comprising a light system configured to emit a light that illuminates a surface of a tray table in both a deployed position for passenger lighting and a stored position for disinfection.

Yet another embodiment of the present disclosure provides a method for illuminating a surface of a tray table. A position of the tray table is detected. A light is emitted that illuminates the surface of the tray table in which the light has a first set of characteristics for passenger lighting in response to the tray table being in a deployed position. The light is emitted that illuminates the surface of the tray table in which the light has a second set characteristics for disinfecting the surface of the tray table in response to the tray table being in a stored position.

Still another embodiment of the present disclosure provides a passenger seat for an aircraft. The passenger seat comprises a seat frame; a seat cushion; arm rests; a backrest; a tray table; and an automatic tray table disinfection system. The automatic tray table disinfection system is configured to emit a light that illuminates a surface of the tray table in a stored position. The light has a set of characteristics that disinfects the surface of the tray table.

The features and functions can be achieved independently in various embodiments of the present disclosure or may be combined in yet other embodiments in which further details can be seen with reference to the following description and drawings.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings show different illustrative examples of automatic tray disinfection systems.

FIG. 1 is an illustration of an exposed view of aircraft in accordance with an illustrative embodiment;

FIG. 2 is an illustration of a block diagram of a disinfection environment in accordance with an illustrative embodiment;

FIG. 3 is an illustration of an automatic tray table disinfection system for passenger seats in accordance with an illustrative embodiment;

FIG. 4 is an illustration of light sources at a top end of a tray table in accordance with an illustrative embodiment;

FIG. 5 is an illustration of light sources emitting light beams on a tray table in accordance with an illustrative embodiment;

FIG. 6 is an illustration of light sources emitting light beams at a top end of a tray table in accordance with an illustrative embodiment;

FIG. 7 is an illustration of light sources and a tray table in accordance with an illustrative embodiment;

FIG. 8 is an illustration of light sources at a top end of a tray table in accordance with an illustrative embodiment;

FIG. 9 is an illustration of light sources emitting light beams on a tray table in accordance with an illustrative embodiment;

FIG. 10 is an illustration of a light source at a top end of a tray table in accordance with an illustrative embodiment;

FIG. 11 is an illustration of a light source emitting a light beam on a tray table in accordance with an illustrative embodiment;

FIG. 12 is an illustration of a light source around the edge of a tray table in accordance with an illustrative embodiment;

FIG. 13 is an illustration of a light strip emitting a light on a tray table in accordance with an illustrative embodiment;

FIG. 14 is an illustration of disinfecting a tray table in accordance with an illustrative embodiment;

FIG. 15 is an illustration of providing passenger lighting in accordance with an illustrative embodiment;

FIG. 16 is an illustration of an automatic tray table disinfection system for a passenger seat in accordance with an illustrative embodiment;

FIG. 17 is an illustration of beam shapes in accordance with an illustrative embodiment;

FIG. 18 is an illustration of light distribution for light propagation between a light source and a reflector in accordance with an illustrative embodiment;

FIG. 19 is an illustration of a system flow diagram in accordance with an illustrative embodiment;

FIG. 20 is an illustration of a flowchart of a process for disinfecting a tray table in accordance with an illustrative embodiment;

FIG. 21 is an illustration of a flowchart of a process for emitting light in accordance with an illustrative embodiment;

FIG. 22 is an illustration of a flowchart of a process for emitting light in accordance with an illustrative embodiment;

FIG. 23 is an illustration of a flowchart of a process for heating a surface of a tray table in accordance with an illustrative embodiment;

FIG. 24 is an illustration of a flowchart of a process for emitting light on a surface of a tray table in accordance with an illustrative embodiment;

FIG. 25 is an illustration of a flowchart of a process for emitting light in accordance with an illustrative embodiment;

FIG. 26 is an illustration of a flowchart of a process for illuminating a surface of a tray table in accordance with an illustrative embodiment;

FIG. 27 is an illustration of a block diagram of an aircraft manufacturing and service method in accordance with an illustrative embodiment; and

FIG. 28 is an illustration of a block diagram of an aircraft in which an illustrative embodiment may be implemented.

### DETAILED DESCRIPTION

The illustrative embodiments recognize and take into account one or more different considerations as described herein. Manual disinfection of tray tables using wipes for disinfection can be prone to inconsistent disinfection of tray tables.

Another solution for disinfecting tray tables can involve inflight disinfection using an overhead reading light with a blue wavelength. However, this solution is impractical because the wavelength of light needed is not practical because blue light interferes with passenger comfort. Passengers often prefer soft white light.

Yet another solution involves using tray table covers. These tray table covers can provide a barrier between the germs and the passenger. These covers are often disposable and can result in increased unnecessary waste material and increased cost.

The illustrative examples provide a number of

different configurations for light systems that can emit light to illuminate the tray table in a manner that disinfects the tray table. As used herein, “a number of” when used with reference to items, means one or more items. For example, “a number of operations” is one or more operations.

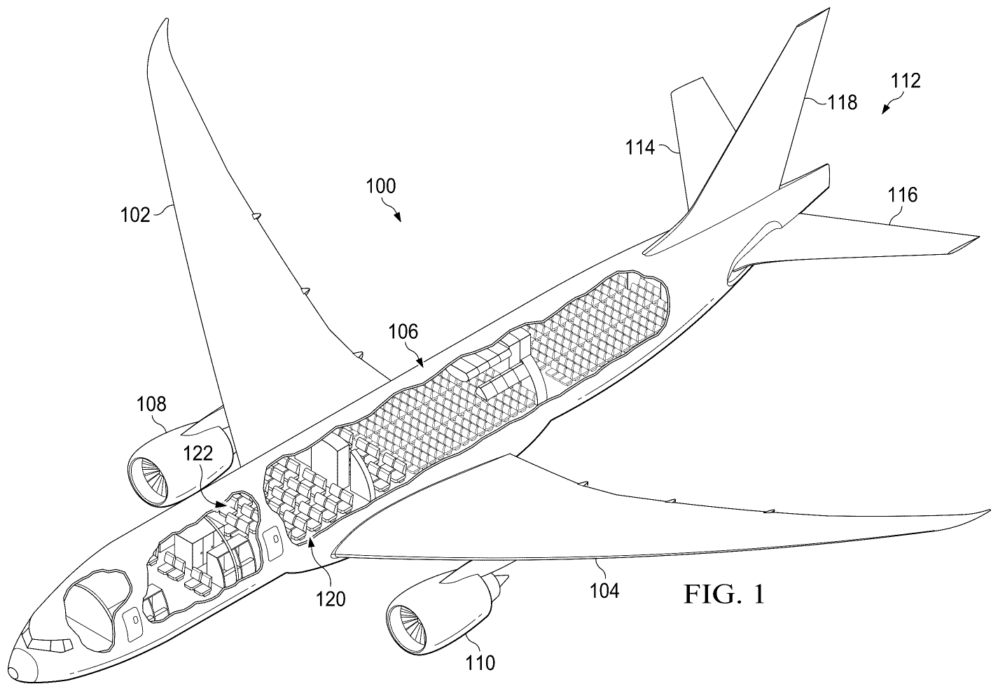

With reference now to the figures, and in particular, with reference to FIG. 1, an illustration of an exposed view of aircraft is depicted in accordance with an illustrative embodiment. In this illustrative example, commercial airplane 100 has wing 102 and wing 104 attached to body 106. Commercial airplane 100 includes engine 108 attached to wing 102 and engine 110 attached to wing 104.

Body 106 has tail section 112, horizontal stabilizer 114, horizontal stabilizer 116, and vertical stabilizer 118 are attached to tail section 112 of body 106.

In this exposed view of commercial airplane 100, passenger seats 120 can be seen within passenger cabin 122. In this example, passenger seats 120 has tray tables (not shown). Commercial airplane 100 is an example of an aircraft in which an automatic tray table sterilization system can be implemented for tray tables for passenger seats 120 in accordance with an illustrative embodiment.

In this example, the automatic tray table system for the tray tables for passenger seats 120 includes a light system that emits light that illuminates surfaces of tray tables for these passenger seats. The light emitted by the automatic tray table system has characteristics that disinfects surfaces of the tray tables when the tray tables are in the stored position.

In these examples, disinfection means that microorganisms are deactivated or destroyed. In these illustrative examples, the microorganisms can be at least one of a bacteria, a virus, a mold, a protozoa and other organisms that are too small to be seen with the naked eye.

A microorganism can be unicellular or multicellular. In many cases, microorganisms are pathogenic and can cause illnesses. For example, microorganisms can cause respiratory illnesses in air travelers. Different illnesses that can cause include at least one of human parainfluenza virus and influenza virus. Other pathogens included adenovirus, non-SARS human CoV, rhinovirus, human metapneumovirus, respiratory syncytial virus, mycoplasma pneumoniae, and Legionella species.

As used herein, the phrase “at least one of,” when used with a list of items, means different combinations of one or more of the listed items can be used, and only one of each item in the list may be needed. In other words, “at least one of” means any combination of items and number of items may be used from the list, but not all of the items in the list are required. The item can be a particular object, a thing, or a category.

For example, without limitation, “at least one of item A, item B, or item C” may include item A, item A and item B, or item B. This example also may include item A, item B, and item C or item B and item C. Of course, any combination of these items can be present. In some illustrative examples, “at least one of” can be, for example, without limitation, two of item A; one of item B; and ten of item C; four of item B and seven of item C; or other suitable combinations.

Disinfection of the tray tables can occur in response to at least one of light or heat on the surface of the tray tables. In these examples, the heat can be generated by light.

The illustration of commercial airplane 100 is provided as an example and not meant to limit the manner in which other illustrative examples can be implemented. For example, the automatic tray table disinfection system can be implemented in other types of aircraft other than commercial airplanes. For example, the automatic tray table disinfection system can be used with a passenger seat in a personal air vehicle, a rotor aircraft, or other type of aircraft in which passenger seats may be used.

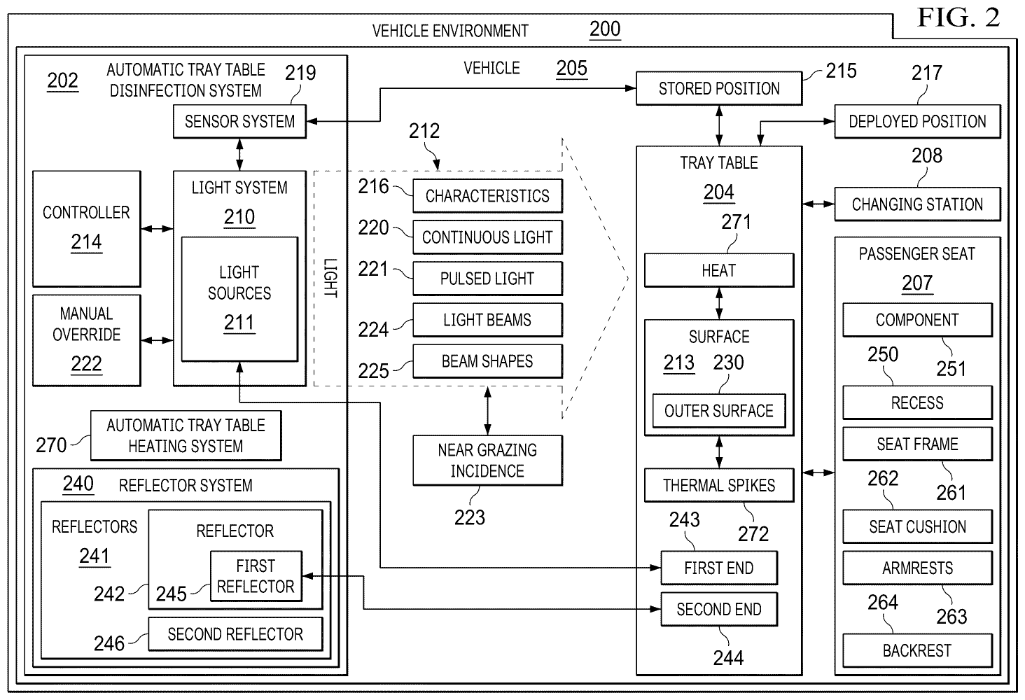

With reference now to FIG. 2, an illustration of a block diagram of a disinfection environment is depicted in accordance with an illustrative embodiment. In vehicle environment 200, automatic tray table disinfection system 202 can operate to disinfect tray table 204 in vehicle 205. In these examples, tray table 204 is for one of a passenger seat 207, a changing station 208, or other monument in the vehicle that has a tray table.

In one illustrative example, tray table 204 is connected to passenger seat 207 located in vehicle 205. Commercial airplane 100 in FIG. 1 is an example of one implementation for vehicle 205.

Vehicle 205 can take a number of different forms. For example, vehicle 205 can be selected from a group comprising a mobile platform, an aircraft, a commercial airplane, a cargo airplane, a rotorcraft, a tilt-rotor aircraft, a tilt wing aircraft, a vertical takeoff and landing aircraft, an unmanned aerial vehicle, an artificial intelligence controlled vehicle, a drone, an electric vertical takeoff and landing vehicle, a personal air vehicle, a surface ship, a tank, a personnel carrier, a train, a spacecraft, a crewed spacecraft, a space plane, a submarine, a bus, and an automobile.

In this example, passenger seat 207 is comprised of a number of different components. Passenger seat 207 can be comprised of seat frame 261, seat cushion 262, armrests 263, and backrest 264. In this example, passenger seat 207 also comprises tray table 204.

Automatic tray table disinfection system 202 can disinfect surface 213 of tray table 204. This disinfection can be performed when tray table 204 is in stored position 215. In this example, stored position 215 is a position in which tray table 204 is placed for storage when not being used. In other words, tray table 204 is not partially open or partially closed when in stored position 215.

In this illustrative example, automatic tray table disinfection system 202 comprises a number of different components. As depicted, automatic tray table disinfection system 202 includes light system 210 and controller 214.

In this example, light system 210 comprises a number of light sources 211. In this example, the number of light sources 211 can be selected from at least one of a light emitting diode, a laser, an ultraviolet lamp, a plasma lamp, a xenon lamp, or other suitable light source that can generate light with disinfecting properties. Light 212 emitted by light system 210 can be selected from at least one of an ultraviolet light, an ultraviolet-C light, ultraviolet-A light, ultraviolet-B light, infrared light, or blue light.

Light system 210 is configured to emit light 212 that illuminates surface 213 of tray table 204 in stored position 215. In this example, light 212 has a set of characteristics 216 that disinfects surface 213 of tray table 204. The set of characteristics 216 can be selected from at least one of a wavelength or power that disinfects the surface 213 of tray table 204.

Also, light system 210 can emit light 212 as at least one of a continuous light 220 or pulsed light 221. Pulsed light 221 can increase the effectiveness of disinfection because of high peak pulse power that can disrupt living organisms. This disruption can be an ionization effect or thermal effect.

Further in this illustrative example, light 212 can be a number of light beams 224 with a number of beam shapes 225. A number of light beams 224 can be selected to provide a desired level of coverage on surface 213 of tray table 204. Beam shapes 225 can take a number of different forms. For example, beam shapes 225 can have cross sections that are round, elongated, elliptical, linear, or other shapes depending on uniformity of light intensity that can be achieved on surface 213 of tray table 204.

When more than one light beam is present in the number of light beams 224, these different light beams can be of different types of light. For example, light 212 for one light beam can be ultraviolet-B light while light 212 for another light beam can be infrared light. In this illustrative example, light 212 disinfects surface 213 tray table 204.

In one illustrative example, light 212 generates heat that disinfects surface 213. In other illustrative examples, other characteristics of light 212 can disinfect surface 213. For example, a wavelength can be selected for light 212 that disinfects surface 213.

In this illustrative example, light 212 can travel in a number of different ways to disinfect surface 213 of tray table 204. For example, light system 210 can emit light 212 directly on outer surface 230 of tray table 204.

In another example, tray table 204 is transparent. With this illustrative example, light system 210 emits light 212 through tray table 204 such that light 212 illuminates surface 213 of tray table 204.

Automatic tray table disinfection system 202 can also include reflector system 240 comprising a number of reflectors 241. In this example, reflector system 240 is an optional component that can aid in the disinfection process by reflecting light 212. This reflection of light 212 results in additional exposure of surface 213 of tray table 204 to light 212.

For example, reflector 242 in the number of reflectors 241 can be located at an opposite end of the tray table 204 that is opposite to an end at which the light system emits light 212. In other words, light 212 can be emitted from light system 210 in a direction towards reflector 242.

In one illustrative example, passenger seat 207 has recess 250 in component 251 of passenger seat 207. Tray table 204 can be stored within recess 250 in stored position 215. This component can be selected from a group comprising a seatback, an armrest, and other components in which tray table 204 can be stored.

In one example, a number of light sources 211 at first end 243 of tray table 204 emits light 212 to illuminate surface 213 of tray table 204. Reflector 242 can be located at second end 244 of tray table 204. This reflector reflects light 212 back towards the number of light sources 211.

Further, reflector 242 can be first reflector 245. Second reflector 246 in the number of reflectors 241 can be located in recess 250. With this example, second reflector 246 has a reflector surface that faces surface 213 of tray table 204 in stored position 215.

In this illustrative example, controller 214

controls the operation of light system 210. For example, controller 214 can cause light system 210 to emit light 212 when tray table 204 is in stored position 215.

Sensor system 219 can be used to detect when tray table 204 is in stored position 215. Sensor system 219 can comprise at least one of a position sensor, a switch for tray table 204, or some other suitable sensor that can detect when tray table 204 is in stored position 215.

For example, a position sensor can detect angular displacement of tray table 204 for tray table 204 in stored position 215. The position sensor can be, for example, an optical encoder, a capacitive displacement sensor, an inductive proximity sensor, an ultrasonic position sensor, a Hall effect sensor, a light sensor, or other type of sensor. When a switch is used, the switch can be changed to a different position in response to placing tray table 204 into stored position 215.

In this illustrative example, control of light system 210 is performed automatically by controller 214. In other words, the disinfection performed by emitting light 212 occurs in response detecting tray table 204 in stored position 215. In other words, a human operator does not need to activate light system 210. This activation occurs automatically in response to tray table 204 being placed into stored position 215.

Controller 214 can take a number of different forms. For example, controller 214 can be hardware. This hardware can be selected from at least one of a circuit system, a switch, an integrated circuit, an application-specific integrated circuit (ASIC), a programmable logic device, a processor unit or some other suitable type of hardware configured to perform a number of operations. With a programmable logic device, the device can be configured to perform the number of operations. The device can be reconfigured at a later time or can be permanently configured to perform the number of operations. Programmable logic devices include, for example, a programmable logic array, a programmable array logic, a field-programmable logic array, a field-programmable gate array, and other suitable hardware devices. Additionally, the processes can be implemented in organic components integrated with inorganic components and can be comprised entirely of organic components excluding a human being. For example, the processes can be implemented as circuits in organic semiconductors.

For example, controller 214 can comprise a switch that causes light system 210 to emit light 212 when tray table 204 is placed into stored position 215. In other examples, controller 214 can be a programmable logic device that causes light 212 to be emitted in response to detecting that tray table 204 has been opened and closed more than once. Further, controller 214 can also control the emission of light 212 such that disinfection occurs as a series of steps for periods of time.

In another example, controller 214 can be a number of processor units that is in communication with other components in vehicle 205. The processor unit can run program instructions to control the operation light system 210. The number of processor units can be at least one of a single core processor, a dual-core processor, a multi-processor core, a general-purpose central processing unit (CPU), a graphics processing unit (GPU), a digital signal processor (DSP), or some other type of processor unit. For example, when vehicle 205 takes the form of an aircraft, controller 214 can communicate with other components in the aircraft to determine when the aircraft has no passengers on board, for example, when the aircraft is parked or flown without passengers.

In these examples, controller 214 controls light system 210 to emit light 212 in a number of different ways under the control of controller 214. For example, controller 214 can control light system 210 to emit light 212 for a selected period of time in response to tray table 204 being placed into stored position 215.

With this example, controller 214 can include a timer that is implemented using program instructions or a timer hardware circuit that can be set to different periods of time in response to detecting tray table 204 in stored position 215. In this example, a sensor such as a switch that is activated when tray table 204 is in stored position 215 and can be used to automatically cause controller 214 to control light system 210 to emit light 212 for a period of time.

The period of time can be some period of time needed to disinfect surface 213 of tray table 204. This period of time can be selected from empirical data gathered by exposing a test surface to test light having different characteristics. The period of time needed to disinfect the tests surface can be used to set the amount of time that light 212 is emitted by light system 210. Further, the number of characteristics 216 for the test light can be changed to determine changes in the period of time needed to disinfect test surfaces. This and other data from testing performed to disinfect test surfaces can be used to set the period of time for which light system 210 emits light 212.

In another example, light 212 can be emitted while tray table 204 is in stored position 215. In this case, light 212 may be emitted for longer periods of time as compared to using a timer. With this implementation, emission of light 212 by light system 210 can be halted when power is shut off to automatic tray table disinfection system 202.

In another illustrative example, manual override 222 can be present for operating light system 210. With this example, manual override 222 can be at least one of a software control or a hardware control switch that can operate light system 210 without needing controller 214. With manual override 222, light system 210 can be activated in response to input from a human operator. This manual override can be used to turn off or deactivate light system 210.

In these illustrative examples, light 212 can be emitted by light system 210 at surface 213 of tray table 204 using near grazing incidence 223. As the angle of incidence for a light ray in light 212 striking surface 213 increases, the likelihood of this light ray being reflected also increases.

In this example, near grazing incidence 223 is the angle for light 212 that is close to 90° relative to the normal angle of surface 213. In other words, light 212 can travel in a direction that is near parallel to surface 213.

In one illustrative example, surface 213 of tray table 204 is a planar nonporous surface that facilitates reflection of light 212 emitted using near grazing incidence 223 to illuminate surface 213 of tray table 204.

In this illustrative example, near grazing incidence 223 for light 212 can be determined using Fresnel equations. Fresnel equations describe how light 212 behaves when light 212 encounters surface 213. This surface is the boundary between air and tray table 204.

These equations can describe the proportions of light 212 that are reflected and transmitted based on the angle of incidence and the polarization of light 212. As the angle of incidence of light 212 approaches 90 degrees (at grazing incidence), these equations predict that the reflectance for both s-polarized (perpendicular) and p-polarized (parallel) light approaches 100%. As a result, nearly all light 212 is reflected.

The Fresnel equations can be used to determine the fraction of light transmitted through an interface. These equations can also be used to determine the fraction of light reflected by an interface such as surface 213. For light moving from air to a solid, the fraction of light 2) increases the closer the angle of incidence approaches 90 degrees.

In another illustrative example, automatic tray table disinfection system 202 can be any form of automatic tray table heating system 270. With this example, light system 210 can emit light 212 that illuminates surface 213 of tray table 204 in stored position 215. Light 212 has a set of characteristics 216 that heats surface 213 of tray table 204 in response to light 212 hitting surface 213. This heating of surface 213 using light 212 is performed such that surface 213 of tray table 204 becomes disinfected. In other words, heat 271 generated on surface 213 of tray table 204 is high enough to disinfect surface 213 of tray table 204.

In one illustrative example, light 212 is pulsed light 221. With this example, pulsed light 221 causes thermal spikes 272 such that temperature increases are greater on the surface 213 of tray table 204 as compared to other parts of tray table 204. The thermal spikes enable local heating and avoid adverse effects of bulk heating, such as material degradation, material outgassing, and wasted energy. Further, thermal spikes can also be more effective at disinfecting than light for some viruses. In some cases, both light and thermal spikes can be used for viruses that may be more difficult to destroy.

As a result, heat 271 disinfects surface 213 of tray table 204.

In yet another illustrative example, automatic tray table disinfection system 202 can have a dual function. This system can provide passenger lighting in addition to disinfecting surface 213 of tray table 204.

Controller 214 can control light system 210 to emit light 212 that illuminates surface 213 of tray table 204 in both deployed position 217 and stored position 215. Light 212 is emitted for passenger lighting in deployed position 217 and for disinfection in stored position 215. With this example, light 212 has a first set of characteristics 216 for passenger lighting in deployed position 217 and a second set of characteristics 216 in stored position 215 for disinfecting surface 213 of tray table 204.

Thus, the illustrative examples provide a method, apparatus, and system for sterilizing surfaces of tray tables.

In the illustrative examples, a disinfection system uses light having characteristics that can disinfect surfaces of the tray tables. The light is turned on automatically in response to the tray table being placed into a stored position. When the tray table is moved out of the stored position, the light is no longer transmitted. In some examples, transmission of light can also be halted after a period of time that is selected to be sufficient to disinfect the surface of the tray table.

The illustration of vehicle environment 200 in FIG. 2 is not meant to imply physical or architectural limitations to the manner in which an illustrative embodiment may be implemented. Other components in addition to or in place of the ones illustrated may be used. Some components may be unnecessary. Also, the blocks are presented to illustrate some functional components. One or more of these blocks may be combined, divided, or combined and divided into different blocks when implemented in an illustrative embodiment.

For example, a number of passenger seats in addition to passenger seat 207 can be present in vehicle 205. With this example, each light source in the number of light sources 211 can be positioned or located to illuminate the tray table or a particular passenger seat.

In some illustrative examples, a controller can be present for each tray table. In another illustrative example, controller 214 can control light sources 211 and light system 210 for all of the tray tables for the passenger seats.

As another example, automatic tray table disinfection system 202 can be considered part of passenger seat 207. For example, the components in automatic tray table disinfection system 202 can be connected to passenger seat 207. With this example, each passenger seat has an associated automatic tray table disinfection system.

When one component is “connected” to another component, the connection is a physical connection. For example, a first component, automatic tray table disinfection system 202, can be considered to be physically connected to a second component, passenger seat 207, by at least one of being secured to the second component, bonded to the second component, mounted to the second component, welded to the second component, fastened to the second component, or connected to the second component in some other suitable manner. The first component also can be connected to the second component using a third component. The first component can also be considered to be physically connected to the second component by being formed as part of the second component, an extension of the second component, or both. In some examples, the first component can be physically connected to the second component by being located within the second component.

Further, although the different illustrative examples show the tray table as being used with a passenger seat, other illustrative examples can be applied to other implementations. For example, the automatic disinfection of tray tables can be used in other locations in an aircraft. As another example, the automatic disinfection system can be used with tray tables for stations in the kitchen area or other food prep area in an aircraft. In another example, a tray table can also be located in the cockpit of the aircraft.

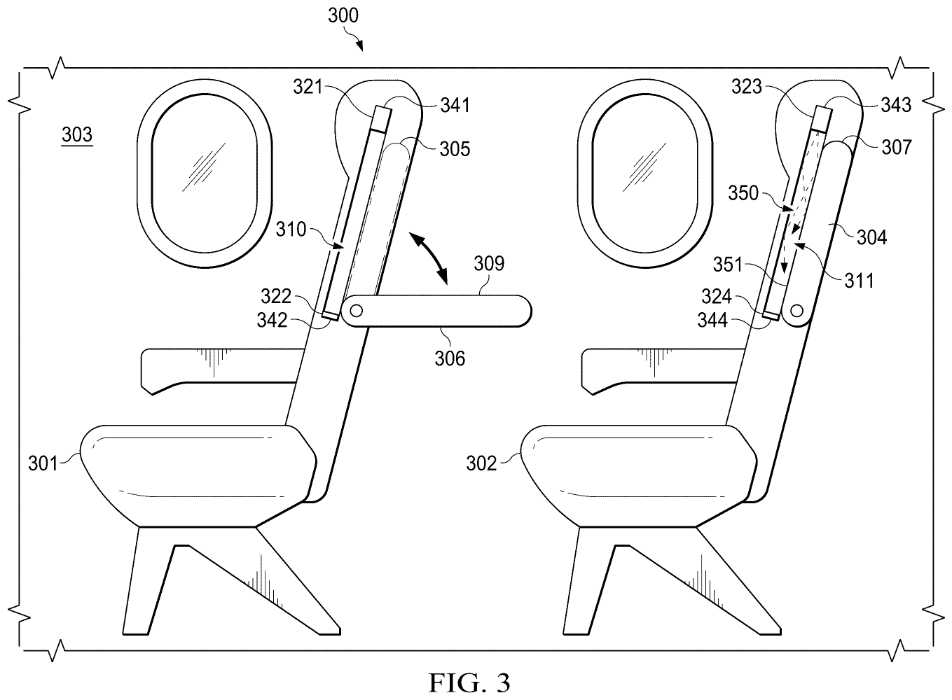

With reference now to FIG. 3, an illustration of an automatic tray table disinfection system for passenger seats is depicted in accordance with an illustrative embodiment. In this example, automatic tray table disinfection system 300 is integrated into passenger seat 301 and passenger seat 302 in cabin 303. Automatic tray table disinfection system 300 is an example of an implementation for automatic tray table disinfection system 202 shown in FIG. 2. These passenger seats are examples of passenger seat 207 in FIG. 2.

As depicted, tray table 309 is connected to the back side of passenger seat 301 and tray table 304 is connected to the back side of passenger seat 302. Tray table 309 is movable between stored position 305 and deployed position 306. As depicted, tray table 309 is in deployed position 306. Tray table 309 can be rotated into recess 310 on the back of passenger seat 301 to be placed in stored position 305. As depicted, tray table 304 is in stored position 307 in recess 311 on the back side of passenger seat 302. In this example, these tray tables are opaque.

As shown in this example, automatic tray table disinfection system 300 comprises blue light source 321, reflector 322, blue light source 323 and reflector 324.

In this example, blue light source 321 is located at first end 341 of recess 310 and reflector 322 is located at second end 342 of recess 310. The location of reflector 322 is at an opposite end of blue light source 321 at first end 341.

Further in this example, blue light source 323 is located at first end 343 of recess 311 and passenger seat 302 and reflector 324 is located at second end 344 of recess 311 and passenger seat 302. Reflector 324 is at an opposite end of first end 343.

These blue light sources are examples of light sources 211 in FIG. 2. The reflectors are examples of reflectors 241 in FIG. 2.

In this example, blue light sources can operate to emit blue light. Blue light can have a wavelength from about 400 nm to about 500 nm. in these examples, a wavelength of 405 nm can be selected for purposes of antimicrobial instruction and general disinfecting purposes. This wavelength can also be referred to as near UV blue lights. Other wavelengths can also be used such as 420 nm to 450 nm in which the blue light damages microbial cells while disinfecting the tray tables.

In this example, blue light source 321 is not emitting a blue light because tray table 309 is not in stored position 305. In other words, blue light is not emitted when tray table 309 is at any position other than stored position 305 in this example.

As depicted, blue light source 323 emits blue light 350 in a manner that reflects off of surface 351 of tray table 304 in stored position 307. Blue light 350 disinfects surface 351 of tray table 304.

Blue light 350 can be emitted until tray table 304 is moved out of stored position 307. In other examples, blue light 350 can be emitted for a period of time while tray table 304 is in stored position 307.

This period of time can be selected as one that results in disinfection of surface 351 of tray table 304.

In this example, these disinfection operations are performed automatically when a tray table is in a stored position. Thus, tray table 309 and tray table 304 can be automatically disinfected in response to these tray tables being placed into a stored position. As a result, blue light can disinfect contaminants such as bacteria when a tray table is in a stored position.

In these examples, the disinfection of the tray tables can be performed manually. In other words, each tray table is closed to the stored position, the blue light is emitted to perform disinfection. The blue light to be emitted as long as the tray table is in the stored position or for some time sufficient to disinfect the tray table.

A tray table may be open to close multiple times during flight. Each time the tray table is opened, additional microorganisms can be deposited on the surface of the tray table. Thus, this section is formed each time the tray tables close. In some cases, the tray table may not be close long enough for the surface to be fully disinfected. As result, opening closing tray table results in an incremental disinfection in which some portion of the microorganisms are destroyed each time the tray table is closed. As a result, each time the tray table was closed a greater percentage of disinfection may occur prior to the tray table being open again.

For example, At 1:20 PM, the tray table is closed play for tray and a stored position. As result, the blue light source is turned on to emit blue light. At 1:25 PM, the tray table is opened, causing the blue light source to be turned off. As a result, between the times 1:20 pm and 1:25 pm, 20% disinfection of microorganisms may occur from the surface of the tray table. In this example, at 2:03 PM, the tray table placed into the stored position and he blue light source is turned on to emit a blue light for disinfection. At 2:33 PM, the tray table is opened so the blue light source is turned off. As a result, 70% disinfection of microorganisms on the surface of the tray table occur between 2:03 pm and 2:33 pm.

Further in this example, at 2:40 PM, the tray table is placed into the stored position in the blue light source is turned on to perform further disinfection of the surface of the tray table. This tray table remains in the stored position for the remainder of the flight, ending at 9:00 PM. In this example, exposure of the surface to blue light for an hour destroys 90% of the microorganisms. As a result, the blue light beam turns off after one hour of exposure time at 3:40 PM even though the flight does not end until 9:00 PM. A disinfection of the surface of the tray table that destroys 90% of the microorganisms may be considered to be sufficient. Thus, the disinfection of the tray table is performed incrementally mentally rather than entirely during the time during which the tray is closed.

In some illustrative examples, between periods of disinfection (stored position), additional microorganisms may be deposited onto the surface of the tray table. For example, the time during at least one of between 1:25 pm and 2:03 PM or between 2:33 pm and 2:40 PM may result in microorganisms being deposited on the tray table while the tray table is opened and not in the stored position. Thus, in some cases disinfection may not reach 90% but the amount of microorganisms present on the surface of the tray table is much lower than without using an automatic tray table disinfection system which reduces the germ count compared to a case without the invention.

The illustration of automatic tray table

disinfection system 300 is provided as one example and is not meant to limit the manner in which this disinfection system can be implemented in other examples. For example, the reflectors are shown at the edge of the recesses opposite to light sources. In other illustrative examples, a reflector can extend the entire length of the insert in the recess of the passenger seat for the tray table without blocking the light. With this example, the reflector has a top, bottom, and sides with an opening for the light to be emitted to perform disinfection. In other words, the reflector may be an open ended rectangular housing covering the tray table when the tray stable is in a stored position.

In another example, other types of light can be used in addition to or in place of blue light. For example, a near infrared light can be used in addition to or in place of the blue light. When a near infrared light is used in addition to blue light, another source that is configured to emit near infrared light can be used in addition to the blue light sources in these examples.

As another example, the tray tables in this figure can be transparent or translucent in other examples. With a transparent or translucent tray table, the light can be emitted through the interior of the tray table in any manner that causes the light to reflect off the surface of the tray tables that are to be disinfected resulting in disinfecting the surfaces of the tray tables.

In still another illustrative example, tray table 309 and tray table 304 can be placed into a stored position in an armrest instead of in a back portion of passenger seat 301 and passenger seat 302. Further, other components can be present but not shown in this example such as a controller to control the operation of blue light source 321 and blue light source 323.

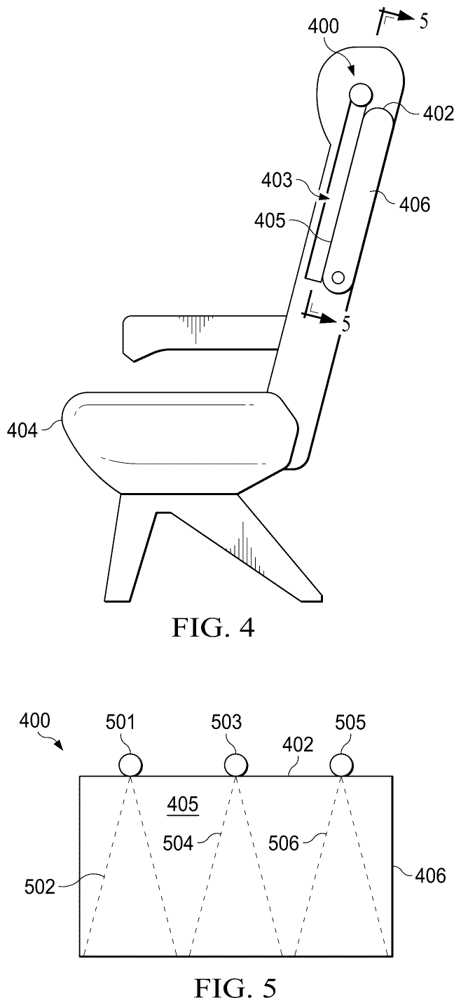

With reference now to FIGS. 4-23, illustrations of light source locations for an automatic tray table disinfection system is depicted in accordance with an illustrative embodiment. These figures show examples of different locations for light sources that can be used to disinfect the tray table. Further, these figures also depict beam shapes that can be used at the different locations.

Turning first to FIG. 4, an illustration of light sources at a top end of a tray table is depicted in accordance with an illustrative embodiment. As depicted, light sources 400 are located at the top end 402 of tray table 406 in a stored position in recess 403 in passenger seat 404. These light sources are positioned at top end 402 in recess 403 such that light emitted by these light sources hit surface 405 of tray table 406 while traveling to the opposite end of recess 403. The light can reflect or bounce off the surface 405.

Next, in FIG. 5, an illustration of light sources emitting light beams on a tray table is depicted in accordance with an illustrative embodiment. This view is a cross-sectional view of light sources 400 and tray table 406 taken along lines 5-5 in FIG. 4.

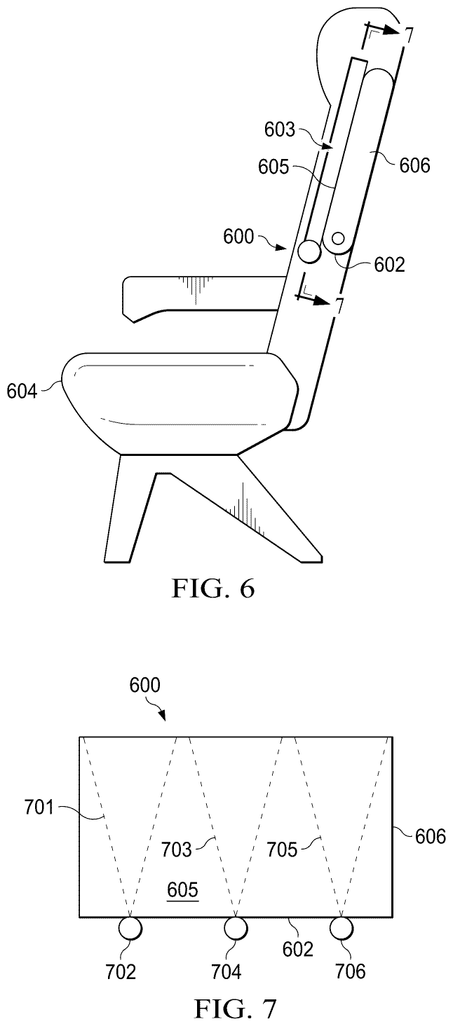

In this figure, light in the form of light beam 502 from light source 501 in light sources 400; light beam 504 from light source 503 in light sources 400; and light beam 506 from light source 505 in light sources 400 are shown. In this example, the light beams have beam shapes that are conical shapes. In some examples, light beams can also have a cross section that is an elliptical shape when this shape enables more even distribution of light as compared to conical shape. With reference next to FIG. 6, an

illustration of light sources emitting light beams at a top end of a tray table is depicted in accordance with an illustrative embodiment. As depicted, light sources 600 are located at the bottom end 602 of tray table 606 in a stored position in recess 603 in passenger seat 604. These light sources are positioned at bottom end 602 in recess 603 such that light emitted by these light sources hit surface 605 of tray table 606 while traveling to the other end of recess 603. The light can reflect or bounce off the surface 805.

Next in FIG. 7, an illustration of light sources and a tray table is depicted in accordance with an illustrative embodiment. This view is a cross-sectional view of light sources 600 and tray table 606 taken along lines 7-7 in FIG. 6.

As depicted, light in the form of light beam 701 from light source 702 in light sources 600; light beam 703 from light source 704 in light sources 600; and light beam 705 from light source 706 in light sources 600 are shown. In this illustrative example, these light beams have beam shapes that are conical shapes and can travel on or reflect off of surface 605 of tray table 606. In some cases, beam shape can have a cross section that is elliptical depending on the matter in which light is distributed.

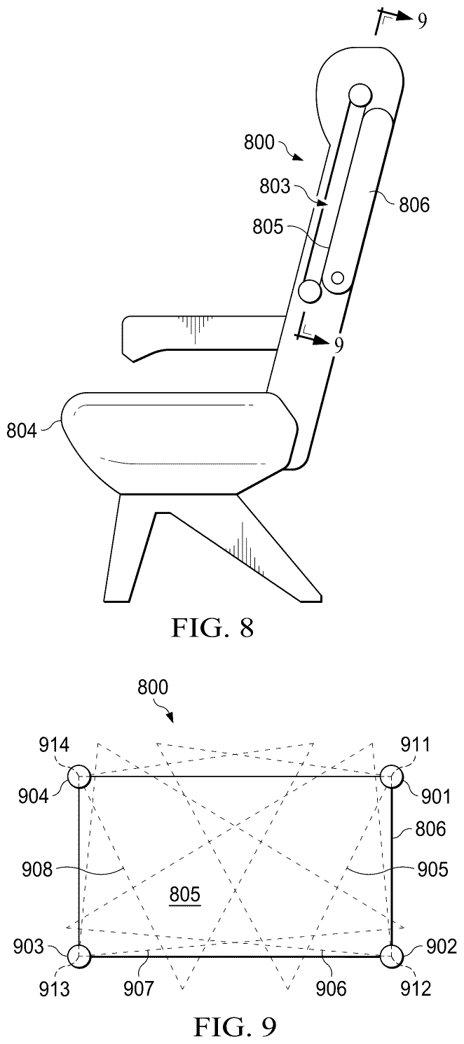

With reference next to FIG. 8, an illustration of light sources at a top end of a tray table is depicted in accordance with an illustrative embodiment. As depicted, light sources 800 are located at the corners of tray table 806 in a stored position in recess 803 in passenger seat 804. These light sources are positioned at the corners in recess 803 such that light emitted by these light sources hit surface 805 of tray table 806 while traveling to the opposite end of recess 803. In other words, the light can reflect or bounce off the surface 805.

Next in FIG. 9, an illustration of light sources emitting light beams on a tray table is depicted in accordance with an illustrative embodiment. This view is a cross-sectional view of light sources 800 and tray table 806 taken along lines 9-9 in FIG. 8.

In this example, light sources 800 include light source 901 at corner 911 of tray table 806, light source 902 at corner 912 of tray table 806, light source 903 at corner 913 of tray table 806, and light source 904 at corner 914 of tray table 806. As depicted, light source 901 emits light beam 905; light source 902 emits light beam 906; light source 903 emits light beam 907; and light source 904 emits light beam 908. In this example, these light beams have beam shapes that are conical shapes and can travel on or reflect off of surface 805 of tray table 806. In some cases, the beam shape can have a cross section that is elliptical depending on the manner in which light is distributed.

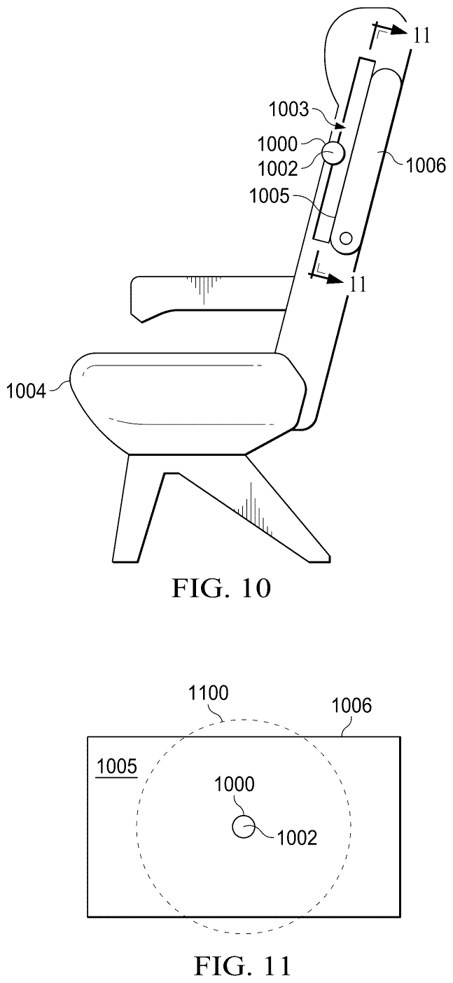

Turning to FIG. 10, an illustration of a light source at a top end of a tray table is depicted in accordance with an illustrative embodiment. As depicted, light source 1000 is positioned at a location that is over center 1002 of tray table 1006 in a stored position in recess 1003 in passenger seat 1004. This light source is positioned over center 1002 of tray table 1006 in recess 403 such that light emitted by light source 1000 hits surface 1005 of tray table 1006.

Next in FIG. 11, an illustration of a light source emitting a light beam on a tray table is depicted in accordance with an illustrative embodiment. This view is a cross-sectional view of light source 1000 and tray table 1006 taken along lines 11-11 in FIG. 10.

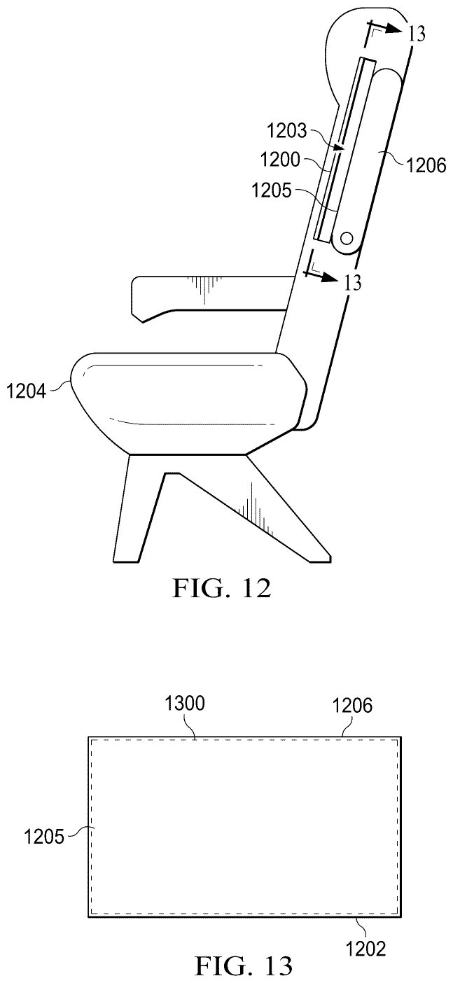

In this depicted example, light source 1000 emits light beam 1100 onto surface 1005 of tray table 1006. In this example, light beam 1100 is a conical shape. Light beam 1100 can have a cross section that is elliptical. With reference now to FIG. 12, an illustration of the light source around the edge of a tray table is depicted in accordance with an illustrative embodiment. As depicted, light strip 1200 is a light source positioned in recess 1203 that extends around edge 1202 of tray table 1206 when tray table 1206 is in a stored position in recess 1203 in passenger seat 1204. This light strip is positioned around edge 1202 of tray table 1206 in recess 403 such that light emitted by light strip 1200 hit surface 1205 of tray table 1206 when tray table 1206 is in the stored position. For example, this light strip can be attached to walls in recess 1203.

Next in FIG. 13, an illustration of a light strip emitting a light on a tray table is depicted in accordance with an illustrative embodiment. This view is a cross-sectional view of light strip 1200 and tray table 1206 taken along lines 13-13 in FIG. 12.

In this depicted example, light strip 1200 emits light 1300 onto surface 1205 of tray table 1206. Light 1300 is emitted from around edge 1202 of tray table 1206. In this example, light 1300 is more evenly distributed as compared to light sources that emit lights from a single or a few different locations.

In this example, light strip 1200 can be positioned along edge 1202 and higher than surface 1205 to emit light 1300 at surface 1205. If tray table 1206 is transparent, then light strip 1200 can be positioned on edge 1202 to emit light 1300 through tray table 1206 to hit surface 1205. For example, the light is emitted such that the light reflects or bounces off of surface 1205.

The illustration of the different light source locations and light beam shapes have been shown as an example and are not meant to limit the manner in which another list of examples can be implemented. For example, only a single light source is shown for tray table 1006 in FIG. 10. In other illustrative examples, one or more light sources may be positioned over other locations for tray table 1006 to cover all of surface 1005 of tray table 1006.

In yet another illustrative example, one or more light sources in addition to light source 1000 can be positioned on the edges, corners, or other locations. Further, other components can be present that are not shown such as a controller to control the operation of the different light sources.

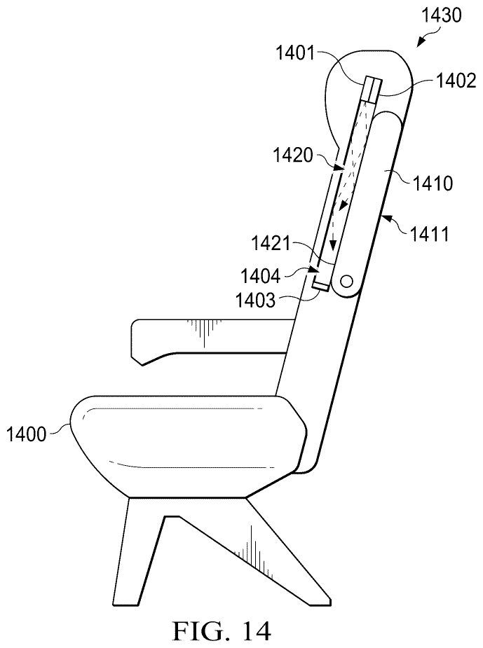

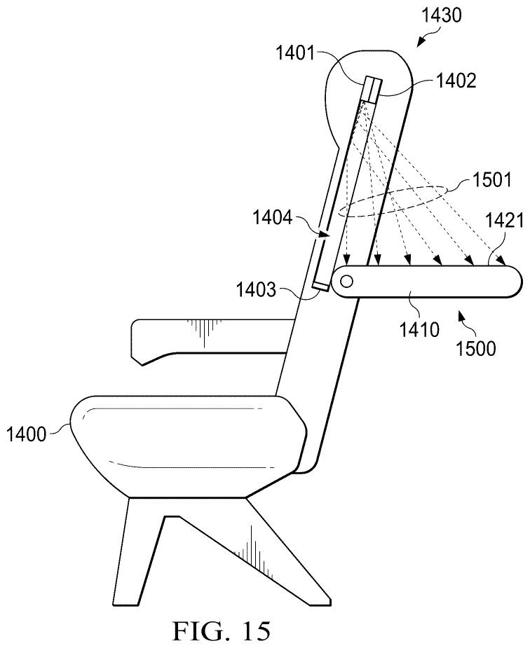

In FIG. 14 and FIG. 15, illustrations of an automatic tray table disinfection system with dual functions are depicted in accordance with an illustrative embodiment. This system can be used to both disinfect a tray table as well as provide passenger lighting.

Turning first to FIG. 14, an illustration of disinfecting a tray table is depicted in accordance with an illustrative embodiment. In this figure, passenger seat 1400 has automatic tray table disinfection system 1430. This system comprises blue light source 1401, passenger light 1402, and reflector 1403. In this example, these three components are located within recess 1404 of passenger seat 1400.

As depicted in this figure, tray table 1410 is in stored position 1411. With tray table 1410 in this position, blue light source 1401 emits blue light 1420 that hits surface 1421 in a manner that blue light 1420 reflects on surface 1421 to reach reflector 1403. Reflector 1403 causes blue light 1420 to reflect back along surface 1421 towards blue light source 1401. This propagation of blue light 1420 results in disinfecting surface 1421 of tray table 1410.

Next in FIG. 15, an illustration of providing passenger lighting is depicted in accordance with an illustrative embodiment. In this figure, tray table 1410 is in deployed position 1500. In this position, passenger light 1402 emits white light 1501 to provide passenger lighting.

In this case, white light 1501 hits surface 1421 of tray table 1410 to provide illumination within the passenger cabin. White light 1501 is selected to provide characteristics suitable for passenger lighting when tray table 1410 is in deployed position 1500. Thus, automatic tray table disinfection system 1430 can emit different types of light depending on the position of tray table 1410.

The illustration of automatic tray table disinfection system 1430 in FIG. 14 and FIG. 15 is provided as an example and not meant to limit the manner in which other examples can be implemented. For example, other components can be present such as a controller to control the operation of blue light source 1401 and passenger light 1402.

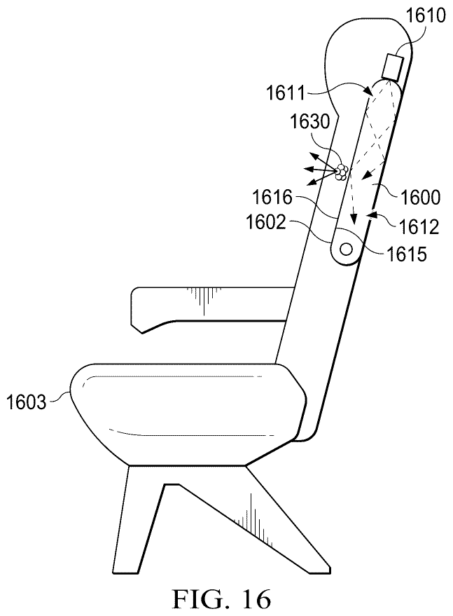

With reference to FIG. 16, an illustration of an automatic tray table disinfection system for a passenger seat is depicted in accordance with an illustrative embodiment. In this illustrative example, tray table 1600 is in a stored position within recess 1602 of passenger seat 1603. Tray table 1600 is a transparent plastic.

With this example, blue light source 1610 transmits blue light 1611 within interior 1612 of tray table 1600. With this example, blue light 1611 is guided using total internal reflection. In response to blue light 1611 hitting internal surface 1615, microbes 1630 on external surface 1616 can be destroyed via the lights evanescent wave. In this manner, the internal reflection of blue light 1611 can be used to disinfect external surface 1616 of tray table 1600.

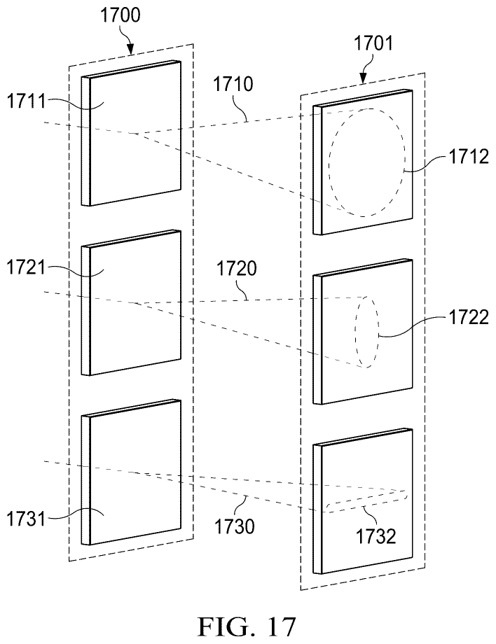

With reference to FIG. 17, an illustration of beam shapes is depicted in accordance with an illustrative embodiment. Lenses 1700 in light sources can be used to generate beam shapes 1701. In this example, beam shapes are shapes for cross-sections of light taken in a direction perpendicular to the direction of propagation along an axis. For example, light 1710 passing through lens 1711 in lenses 1700 results in a beam cross-section that is circular shape 1712. This shape can be used to reduce or eliminate hot spots in light 1710. In other words, localized regions of different intensities can be reduced or eliminated in light 1710.

As another example, light 1720 passing through lens 1721 in lenses 1700 has elliptical shape 1722. In this example, elliptical shape 1722 can be used to control a divergence angle of light 1720. In this example, the spread of light 1720 can be reduced as light 1720 propagates.

In yet another illustrative example, lens 1731 in lenses 1700 causes light 1730 passing through this lens to have linear shape 1732. This linear shape can reduce speckle. Reducing speckle can mean a random granular pattern within light 1730 can be reduced.

This and other types of shaping of light can result in less loss and more even distribution of the light across the surface of the tray table.

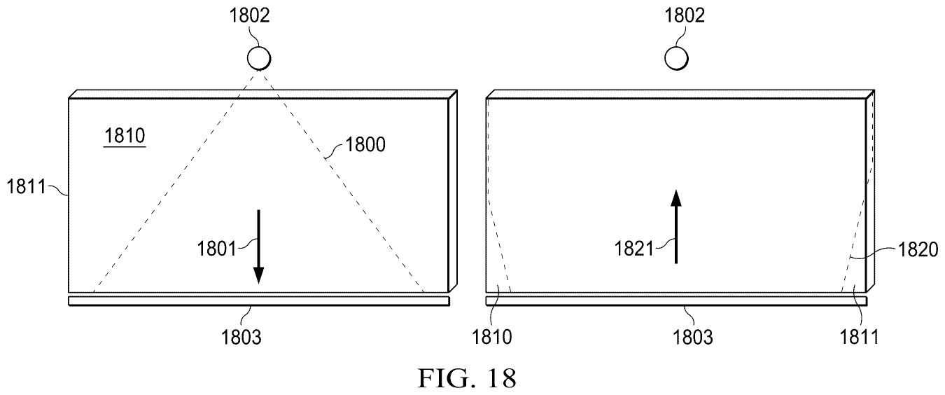

With reference now to FIG. 18, an illustration of light distribution for light propagation between a light source and a reflector is depicted in accordance with an illustrative embodiment. As depicted, light 1800 propagates in the direction of arrow 1801 from light source 1802 towards reflector 1803. The propagation of light 1800 includes light that propagates by at least one of traveling on or reflecting off surface 1810 on tray table 1811.

Light 1800 is reflected on reflector 1803 to result in reflected light 1820 propagating in the direction of arrow 1821. As depicted, reflected light 1820 provides greater coverage on surface 1810 of tray table 1811.

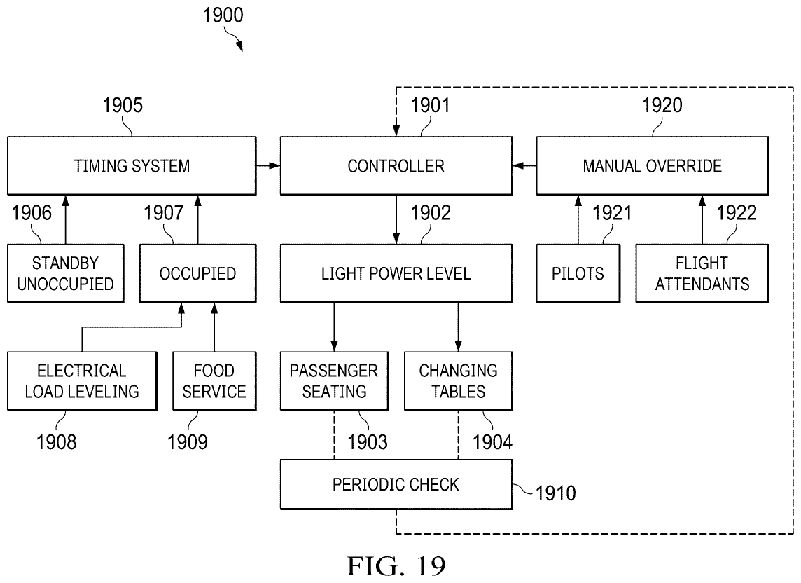

Next in FIG. 19, an illustration of a system flow diagram is depicted in accordance with an illustrative embodiment. In this example, system flow 1900 can be implemented in automatic tray table disinfection system 202 in FIG. 2.

in this example, controller 1901 in system flow 1900 can turn on light sources to perform disinfection of passenger seating 1903 and changing tables 1904 using timing system 1905. Timing system 1905 is used to determine when the disinfection should occur. For example, timing system 1905 can set one or more time periods for disinfecting passenger seats based on whether a particular seat is on standby unoccupied 1906 or occupied 1907.

Timing system 1905 can perform a number of different functions used by controller 1901 to determine when to turn light sources on-and-off for different passenger seats in the aircraft.

For example, timing system 1905 can be used to determine whether particular passenger seats in passenger seating 1903 are standby unoccupied 1906 or occupied 1907. Standby unoccupied 1906 means that a particular passenger seat in passenger seating 1903 is not occupied. This determination can he made based on whether the passenger seat has been assigned to passenger or using a sensor to determine whether a passenger is sitting in the passenger seat.

When a passenger seat in passenger seating 1903 is standby unoccupied 1906, the tray table for that passenger seat does not need to be disinfected. As result, the lighting system for disinfecting that tray table does not turn on when the tray table is in the stored position.

If a passenger seat in passenger seating 1903 becomes occupied, the lighting system is taken out of standby unoccupied 1906 and enters occupied 1907. In this case, timing system 1905 sends a signal to controller 1901 to indicate that the passenger seat is now occupied 1907. In response, controller 1901 causes the lighting source for the tray table for that passenger seat to emit a light to disinfect the tray table response to the tray table being in a stored position.

Thus, timing system 1905 can track which seats are standby unoccupied 1906 and occupied 1907, sending this information to controller 1901 were used by controller 1901 in determining which light sources to be activated when tray tables are in stored positions.

Further, timing system 1905 can also control how long light sources are turned on for tray tables in the stored positions. For example, timing system 1905 can determine when a period of time has passed since a tray table has been in the stored position. An indication that the paired of time has expired can be sent to controller 1901 to cause controller 1901 to turn off the light even though the seat is occupied 1907 and the tray table has been in a stored position.

Thus, timing system 1905 can be used by controller 1901 to determine when a period of time has passed such light no longer needs to be emitted to disinfect a tray table.

When passenger seats are occupied 1907, electrical load leveling 1908 and food service 1909 are taken into account by timing system 1905 when the passenger seats are occupied 1907. For example, if a phase of flight of the aircraft requires a large but temporary amount electrical power, the lighting systems can be turned off even though the tray tables are in a stored position for passenger seats are occupied 1907.

When food service 1909 occurs during flight, the period of time that the lighting system is turned on is increased. For example, if a normal period of time for disinfection is one hour. Time can be increased to two hours when food service 1909 is present. In this manner, increased amount of time can be used to enable disinfecting tray tables, which may become more difficult to disinfect when food is served during the flight.

In causing the light sources to emit light for disinfection, controller 1901 controls light power level 1902 for light sources for passenger seating 1903 and changing tables 1904. Light power level 1902 can be controlled based on the passenger seat occupant. For example, a child assigned to the passenger seat may result in positing more microorganisms than in adults. In this case, tray tables for those passenger seats may use a higher light power level or increase disinfection.

In this example, periodic manual check 1910 can be performed on passenger seating 1903 and changing tables 1904. This periodic check can be used to fine-tune power light levels for different locations in the airplane.

Different factors which can alter the

effectiveness of the disinfection. The effectiveness of light sources such as blue lights can fade over time. As another example, the surface of tray tables may become scuffed in a manner making it more difficult to disinfect the tray tables. Periodic tests of microorganism levels or times needed to disinfect tray tables can be performed to determine whether adjustments are needed to ensure that the disinfection systems for these tray tables are effectively disinfecting the tray tables.

In this illustrative example, manual override 1920 can be used to turn on or turn off the light sources. This manual override can be performed by human operators such as pilots 1921 and flight attendants 1922. This manual override can be performed to turn off power to the light sources for the tray tables when pilots 1921 or flight attendants 1922 determine it is necessary to conserve power.

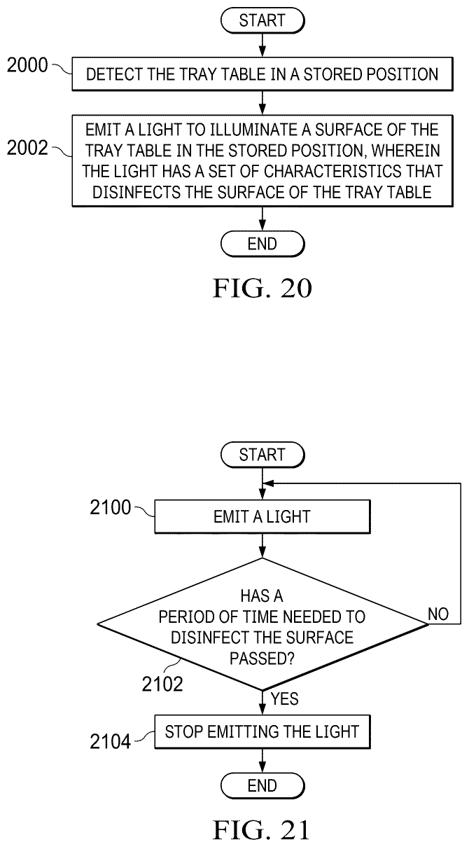

Turning next to FIG. 20, an illustration of a flowchart of a process for disinfecting a tray table is depicted in accordance with an illustrative embodiment. The process in FIG. 20 can be implemented using automatic tray table disinfection system 202 in FIG. 2. This process can be implemented in hardware, software, or both. In one example, the process can be implemented in controller 214 in FIG. 2 using at least one of hardware or software.

The process begins by detecting the tray table in a stored position (operation 2000). The process emits a light to illuminate a surface of the tray table in the stored position, wherein the light has a set of characteristics that disinfects the surface of the tray table (operation 2002). The process terminates thereafter.

In FIG. 21, an illustration of a flowchart of a process for emitting light is depicted in accordance with an illustrative embodiment. This flowchart is an example of an implementation for operation 2002 in FIG. 20.

The process emits a light (operation 2100). A determination is made as to whether a period of time needed to disinfect the surface has passed (operation 2102). If the period of time has not passed, the process returns to operation 2102. Otherwise, the process stops emitting the light (operation 2104). The process terminates thereafter. In this case, disinfection of the surface of the tray table has occurred.

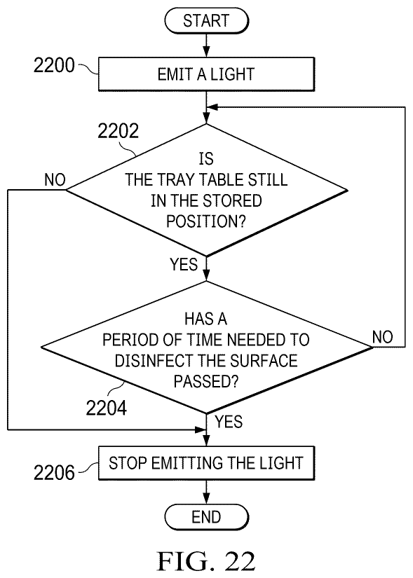

With reference now to FIG. 22, an illustration of a flowchart of a process for emitting light is depicted in accordance with an illustrative embodiment. This flowchart is an example of an implementation for operation 2002 in FIG. 20. This process can be used when the characteristics of the light being emitted are not eye safe or may be disruptive. For example, if darkness is desired in the cabin, it is undesirable to emit light when the tray table is open from the stored position.

The process emits a light (operation 2200). A determination is made as to whether the tray table is still in the stored position (2202). If the tray table is still in the stored position, a determination is made as to whether a period of time needed to disinfect the surface has passed (operation 2204). If the period of time has not passed, the process returns to operation 2202. Otherwise, the process stops emitting the light (operation 2206). In operation 2206, the surface of the tray table has been disinfected and the light no longer needs to be emitted.

With reference again to operation 2202, if the tray table is no longer in the stored position, the process proceeds to operation 2206 to stop emitting the light.

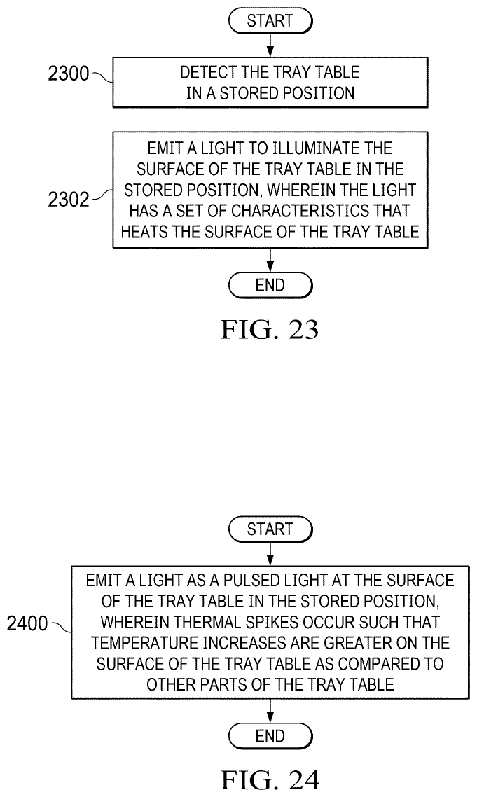

Turning now to FIG. 23, an illustration of a flowchart of a process for heating a surface of a tray table is depicted in accordance with an illustrative embodiment. The process in FIG. 23 can be implemented using automatic tray table disinfection system 202 in FIG. 2. This process can be implemented in hardware, software, or both. In one example, the process can be implemented in controller 214 in FIG. 2 using at least one of hardware or software.

The process detects the tray table in a stored position (operation 2300). The process emits a light to illuminate the surface of the tray table in the stored position, wherein the light has a set of characteristics that heats the surface of the tray table (operation 2302). The process terminates thereafter. In operation 2302, the heating disinfects the surface of the tray table. This heating can be such that disinfection occurs through destroying microorganisms. In this example, operation 2302 can be performed for a period of time that is sufficient to disinfect the surface of the tray table.

Next in FIG. 24, an illustration of a flowchart of a process for emitting light on a surface of a tray table is depicted in accordance with an illustrative embodiment. The flowchart in this process is an example of an implementation for operation 2302 in FIG. 23.

The process emits a light as a pulsed light at the surface of the tray table in the stored position, wherein thermal spikes occur such that temperature increases are greater on the surface of the tray table as compared to other parts of the tray table (operation 2400). The process terminates thereafter.

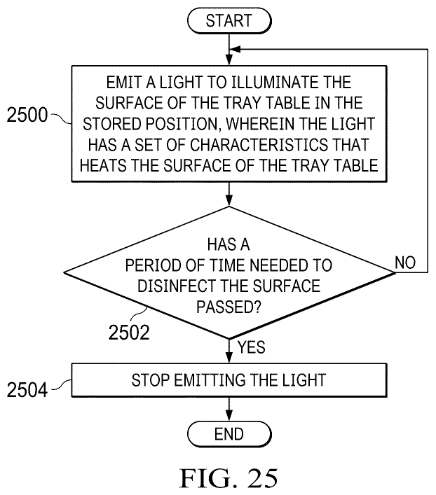

In FIG. 25, an illustration of a flowchart of a process for emitting light is depicted in accordance with an illustrative embodiment. This flowchart is an example of an implementation for operation 2302 in FIG. 23.

The process emits a light to illuminate the surface of the tray table in the stored position, wherein the light has a set of characteristics that heats the surface of the tray table (operation 2500). A determination is made as to whether a period of time needed to disinfect the surface has passed (operation 2502). If the period of time has not passed, the process returns to operation 2500. Otherwise, the process stops emitting the light (operation 2504). The process terminates thereafter. In this case, disinfection of the surface of the tray table through heating has occurred.

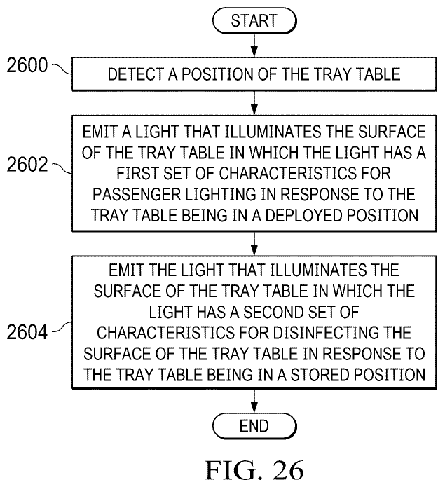

Turning now to FIG. 26, an illustration of a flowchart of a process for illuminating a surface of a tray table is depicted in accordance with an illustrative embodiment. The process in FIG. 26 can be implemented using automatic tray table disinfection system 202 in FIG. 2. This process can be implemented in hardware, software, or both. In one example, the process can be implemented in controller 214 in FIG. 2 using at least one of hardware or software.

The process begins by detecting a position of the tray table (operation 2600). The process emits a light that illuminates the surface of the tray table in which the light has a first set of characteristics for passenger lighting in response to the tray table being in a deployed position (operation 2602).

The process emits the light that illuminates the surface of the tray table in which the light has a second set of characteristics for disinfecting the surface of the tray table in response to the tray table being in a stored position (operation 2604). The process terminates thereafter. In this example, the second set of characteristics are such that the surface of the tray table is disinfected by being illuminated with the light having the second set of characteristics. This operation can be performed for some period of time or can be continuous while the tray table is in the stored position.

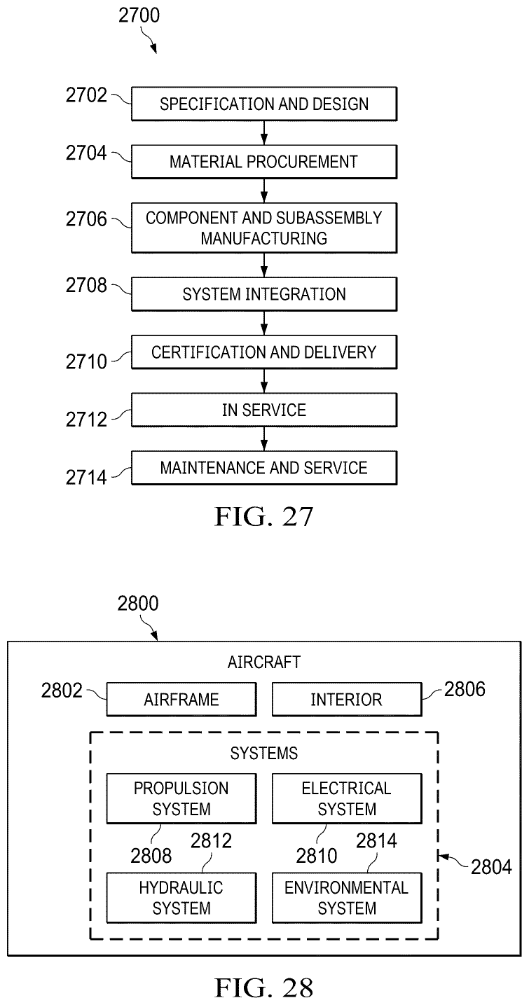

Illustrative embodiments of the disclosure may be described in the context of aircraft manufacturing and service method 2700 as shown in FIG. 27 and aircraft 2800 as shown in FIG. 28. Turning first to FIG. 27, an illustration of a block diagram of an aircraft manufacturing and service method is depicted in accordance with an illustrative embodiment. During pre-production, aircraft manufacturing and service method 2700 may include specification and design 2702 of aircraft 2800 in FIG. 28 and material procurement 2704.

During production, component and subassembly manufacturing 2706 and system integration 2708 of aircraft 2800 in FIG. 28 takes place. Thereafter, aircraft 2800 in FIG. 28 can go through certification and delivery 2710 in order to be placed in service 2712. While in service 2712 by a customer, aircraft 2800 in FIG. 28 is scheduled for routine maintenance and service 2714, which may include modification, reconfiguration, refurbishment, and other maintenance or service.

Each of the processes of aircraft manufacturing and service method 2700 may be performed or carried out by a system integrator, a third party, an operator, or some combination thereof. In these examples, the operator may be a customer. For the purposes of this description, a system integrator may include, without limitation, any number of aircraft manufacturers and major-system subcontractors; a third party may include, without limitation, any number of vendors, subcontractors, and suppliers; and an operator may be an airline, a leasing company, a military entity, a service organization, and so on.

With reference now to FIG. 28, an illustration of a block diagram of an aircraft is depicted in which an illustrative embodiment may be implemented. In this example, aircraft 2800 is produced by aircraft manufacturing and service method 2700 in FIG. 27 and may include airframe 2802 with plurality of systems 2804 and interior 2806. Examples of systems 2804 include one or more of propulsion system 2808, electrical system 2810, hydraulic system 2812, and environmental system 2814. Any number of other systems may be included. Although an aerospace example is shown, different illustrative embodiments may be applied to other industries, such as the automotive industry.

Apparatuses and methods embodied herein may be employed during at least one of the stages of aircraft manufacturing and service method 2700 in FIG. 27.

In one illustrative example, components or subassemblies produced in component and subassembly manufacturing 2706 in FIG. 27 can be fabricated or manufactured in a manner similar to components or subassemblies produced while aircraft 2800 is in service 2712 in FIG. 27. As yet another example, one or more apparatus embodiments, method embodiments, or a combination thereof can be utilized during production stages, such as component and subassembly manufacturing 2706 and system integration 2708 in FIG. 27. One or more apparatus embodiments, method embodiments, or a combination thereof may be utilized while aircraft 2800 is in service 2712, during maintenance and service 2714 in FIG. 27, or both. The use of a number of the different illustrative embodiments may substantially expedite the assembly of aircraft 2800, reduce the cost of aircraft 2800, or both expedite the assembly of aircraft 2800 and reduce the cost of aircraft 2800.

For example, automatic tray table disinfection system 202 can be implemented during at least one of system integration 2708, in service 2712, or maintenance and service 2714. This type of system can be implemented into passenger seating during system integration 2708 in manufacturing of aircraft 2800. Automatic tray table disinfection system 202 can be added as part of maintenance and service 2714. For example, the system can be added during modification, reconfiguration, refurbishment, and other maintenance or service for aircraft 2800.

Additionally, disinfection of tray table surfaces can be performed while aircraft 2800 is in service 2712. This system can reduce the amount of time needed to clean and disinfect passenger cabins through the ability to disinfect tray tables for passenger seats.

The description of the different illustrative embodiments has been presented for purposes of illustration and description and is not intended to be exhaustive or limited to the embodiments in the form disclosed. The different illustrative examples describe components that perform actions or operations. In an illustrative embodiment, a component can be configured to perform the action or operation described. For example, the component can have a configuration or design for a structure that provides the component an ability to perform the action or operation that is described in the illustrative examples as being performed by the component. Further, to the extent that terms “includes”, “including”, “has”, “contains”, and variants thereof are used herein, such terms are intended to be inclusive in a manner similar to the term “comprises” as an open transition word without precluding any additional or other elements.

Many modifications and variations will be apparent to those of ordinary skill in the art. Further, different illustrative embodiments may provide different features as compared to other desirable embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

What is claimed is:

1. An automatic tray table disinfection system comprising:
a light system configured to emit a light that illuminates a surface of a tray table in a stored position, wherein the light has a set of characteristics that disinfects the surface of the tray table.

2. The automatic tray table disinfection system of claim 1 further comprising:
a manual override for operating the light system.

3. The automatic tray table disinfection system of claim 1, wherein the light is emitted at the surface of the tray table surface using a near grazing incidence.

4. The automatic tray table disinfection system of claim 1, wherein the surface of the tray table is a planar nonporous surface that facilitates reflection of the light emitted using a near grazing incidence to illuminate the surface of the tray table.

5. The automatic tray table disinfection system of claim 1, wherein the light comprises a number of light beams with a number of beam shapes.

6. The automatic tray table disinfection system of claim 1 further comprising:
a reflector at an opposite end of the tray table that is opposite to an end at which the light system emits the light.

7. The automatic tray table disinfection system of claim 1 further comprising:
a recess in a component of a passenger seat in which the tray table is stored in the stored position;
wherein the light system comprises:
a number of light sources at a first end of the tray table that emits the light to illuminate the surface of the tray table; and
a reflector at a second end of the tray table that reflects the light back towards the number of light sources.

8. The automatic tray table disinfection system of claim 7, wherein the reflector is a first reflector and further comprising:
a second reflector connected at a location in the recess, wherein the second reflector has a reflector surface that faces the surface of the tray table in the stored position.

9. The automatic tray table disinfection system of claim 7, wherein the component is selected from a group comprising a backrest and an armrest.

10. The automatic tray table disinfection system of claim 1, wherein the set of characteristics is selected from at least one of a wavelength or power that disinfects the surface of the tray table.

11. The automatic tray table disinfection system of claim 1, wherein the light system emits the light as at least one of a continuous light or a pulsed light.

12. The automatic tray table disinfection system of claim 7, wherein the number of light sources is selected from at least one of a light emitting diode, a laser, an ultraviolet lamp, a plasma lamp, or a xenon lamp.

13. The automatic tray table disinfection system of claim 1, wherein the light system emits the light directly on an outer surface of the tray table.

14. The automatic tray table disinfection system of claim 1, wherein the tray table is transparent and wherein the light system emits the light through the tray table such that the light illuminates the surface of the tray table.

15. The automatic tray table disinfection system of claim 1, wherein the light disinfects the surface.

16. The automatic tray table disinfection system of claim 1, wherein the light generates heat that disinfects the surface.

17. The automatic tray table disinfection system of claim 1, wherein the light is selected from at least one of an ultraviolet light, an ultraviolet-C light, an ultraviolet-A light, an ultraviolet-B light, an infrared light, or a blue light.

18. The automatic tray table disinfection system of claim 1, wherein the tray table is for one of a passenger seat and a changing station.

19. A method for disinfecting a tray table, the method comprising:
detecting the tray table in a stored position; and
emitting a light to illuminate a surface of the tray table in the stored position, wherein the light has a set of characteristics that disinfects the surface of the tray table.

20. An automatic tray table heating system comprising:
a light system configured to emit a light that illuminates a surface of a tray table in a stored position, wherein the light has a set of characteristics that heats the surface of the tray table.

21. The automatic tray table heating system of claim 20, wherein the light is a pulsed light and wherein the pulsed light causes thermal spikes such that temperature increases are greater on the surface of the tray table as compared to other parts of the tray table.

22. The automatic tray table heating system of claim 21, wherein the heat disinfects the surface of the tray table.

23. A method for heating a surface of a tray table, the method comprising:
detecting the tray table in a stored position; and
emitting a light to illuminate the surface of the tray table in the stored position, wherein the light has a set of characteristics that heats the surface of the tray table.

24. The method of claim 23, wherein emitting the light comprises:
emitting a light as a pulsed light at the surface of the tray table in the stored position, wherein thermal spikes occur such that temperature increases are greater on the surface of the tray table as compared to other parts of the tray table.

25. The method of claim 23, wherein heating disinfects the surface of the tray table.

26. An automatic tray table lighting system comprising:
a light system configured to emit a light that illuminates a surface of a tray table in both a deployed position for passenger lighting and a stored position for disinfection.

27. The automatic tray table lighting system of claim 26, wherein the light has a first set of characteristics for passenger lighting in the deployed position and a second set characteristics in the stored position for disinfecting the surface of the tray table.

28. A method for illuminating a surface of a tray table, the method comprising:
detecting a position of the tray table;
emitting a light that illuminates the surface of the tray table in which the light has a first set of characteristics for passenger lighting in response to the tray table being in a deployed position; and
emitting the light that illuminates the surface of the tray table in which the light has a second set characteristics for disinfecting the surface of the tray table in response to the tray table being in a stored position.

29. A passenger seat for an aircraft, the passenger seat comprising:
a seat frame;
a seat cushion;
arm rests;
a backrest;
a tray table; and
an automatic tray table disinfection system configured to emit a light that illuminates a surface of the tray table in a stored position, wherein the light has a set of characteristics that disinfects the surface of the tray table.

30. The passenger seat of claim 29, further comprising:
a recess in a back side of the backrest, wherein the tray table is stored in the recess in the stored position.
